(19) United States
(12) Patent Application Publication
(10) Pub. No.: US 2003/0235618 A1
(43) Pub. Date: Dec. 25, 2003

(54) Taste masking spill-resistant formulation

(75) Inventors: Daniel A. Moros, Larchmont, NY (US); Shen Gao, Brampton (CA); Maxine Gay Moldenhauer, Acton (CA)

Correspondence Address: VENABLE, BAETJER, HOWARD AND CIVILETTI, LLP, P.O. BOX 34385, WASHINGTON, DC, 20043-9998, US

(73) Assignee: Taro Pharmaceutical Industries Ltd., Haifa Bay (IL)

(21) Appl. No.: 10/386,938

(22) Filed: Mar. 13, 2003

Related U.S. Application Data
(63) Continuation-in-part of application No. 10/277,083, filed on Oct. 22, 2002, now abandoned.
(60) Provisional application No. 60/330,447, filed on Oct. 22, 2001.

(51) Int. Cl. A61K 31/16; A61K 9/14

(52) U.S. Cl. 424/486; 514/629

(57) ABSTRACT

The invention relates to a taste masking spill-resistant pharmaceutical composition, comprising a spill-resistant formulation with taste masking concentrations of polyethylene glycol (PEG) which is less bitter, sweeter and has better overall flavor than current pharmaceutical compositions, while maintaining advantageous spill-resistant properties.

This application claims the benefit of provisional application U.S. Ser. No. 60/330,447, filed Oct. 22, 2001, and is a continuation-in-part of U.S. Ser. No. 10/277,083, filed on Oct. 22, 2002, both incorporated herein by reference.

### BRIEF DESCRIPTION OF THE INVENTION

The invention relates to a taste masking spill-resistant pharmaceutical composition, comprising a spill-resistant formulation with a taste masking concentration of polyethylene glycol (PEG), which is less bitter, sweeter and has better overall flavor than current pharmaceutical compositions, while maintaining advantageous spill-resistant properties.

### BACKGROUND OF THE INVENTION

Liquid formulations for oral delivery of pharmaceutical agents are desirable because certain patients, such as children and the elderly, are unable to swallow capsules or tablets. However, liquid preparations are messy, require shaking before use, and the measurement of an exact dose is difficult. Additionally, the bitter, unpleasant medicine taste of medicinal compounds is especially noticeable in liquid formulations because of the liquids increased ability to interact with the sense receptors in the mouth.

Pharmaceutically active agents are known to impart a “medicinal”, bitter, sour taste to pharmaceutical formulations. This taste is especially noticeable in liquid formulations due to the long period of time that the liquid allows the active agent to be in contact with taste receptors. Taste masking agents are common in the art. U.S. Pat. No. 5,730,997 to Lienhop et al. describes a taste masking liquid solution of pharmaceutically active agents dispersed in a high osmolarity aqueous solution. The high osmolarity aqueous solution contains high concentrations of sugars, and hydrogenated maltose syrup. U.S. Pat. 5,602,182 to Popli et al. describes the use of a solid polyethylene glycol and an acidic pH to taste mask liquid pharmaceutical compositions. These liquid compositions are spillable, and of relatively low viscosity.

U.S. Pat. No. 6,071,523 to Mehta et al. and U.S. Pat. No. 5,881,926 to Ross describe semi-solid spill-resistant compositions that contain pharmaceutically active agents, and devices for their delivery. These patents do not have a solution for the bitter, medicinal taste that is a common problem of pharmaceutical preparations.

There remains a need for formulations with improved palatability, that have the requisite characteristics of a spill-resistant formulation

### SUMMARY OF THE INVENTION

The invention provides for a palatable, semi-solid, spill-resistant pharmaceutical solution for oral administration comprising (a) from about 0.5 to about 5.0% of acetaminophen or another pharmaceutically active agent; (b) from about 0.18 to about 0.35% of a carbomer; (c) up to about 50% glycerin; (d) from about 5 to about 30% polyethylene glycol; (e) up to about 2% sucralose liquid concentrate; and (f) water. The composition has a pH of between about 5.0 to about 7.5 and a viscosity of between about 6,000 to about 20,000 cps. The taste-masking component of the composition is the PEG in concentrations of between about 10 to about 30% of the total composition.

An embodiment of this invention is that the pharmaceutically active agent of the composition is acetaminophen.

A further embodiment of this invention is that the pharmaceutically active agent of the composition is selected from the group consisting of analgesics, anti-inflammatory agents, anti-histamines, anti-infectives, bronchodilators, cough suppressants, expectorants, decongestants, CNS active agents, anti-convulsants, cardiovascular agents, antineoplastics, cholesterol-lowering agents, anti-emetics, vitamins, minerals, plant extracts and pharmaceutically acceptable salts and esters thereof.

This invention allows for a pharmaceutical composition further comprising at least one pharmaceutically acceptable excipient selected from the group consisting of at least one food dye, masking agent, flavoring agent and antimicrobial agent.

The invention provides a palatable, semi-solid, spill-resistant pharmaceutical solution for oral administration comprising (a) 2.75% acetaminiophen; (b) 0.27% carbomer 939P; (c) up to 50% glycerin; (d) 15% polyethylene glycol 1000; (e) 0.4% sucralose liquid concentrate; and (f) water. The composition has a pH of 6.0 to 7.2 and a viscosity of between 6,000 to 13,000 cps.

A further embodiment of this invention is a unit dosage form for systemic treatment by the oral route of children, which is also convenient for self administration by aging adults, as well as adults with motor problems.

Another embodiment of this invention is the process for preparing a palatable, semi-solid, spill-resistant pharmaceutical suspension comprising (a) dispersing the carbomer in a liquid such as water or propylene glycol until a lump free dispersion is formed; (b) dissolving the acetaminophen into water; (c) mixing the butylparaben into the solution of step (b); (d) heating the polyethylene glycol to 60° C. to 70° C. until it is dissolved; (e) mixing the polyethylene glycol and glycerin into the solution of step (c); (f) mixing the solution of step (e) and solution of step (a) and cooling the mixture to less than 40° C.; (g) mixing the food coloring with water until a clear solution is formed; (h) adding the solution of step (g) with the solution of step (e) mixing with food coloring, masking agents and sucralose liquid concentrate; and (i) titrating the solution of step (g) with a sodium hydroxide solution to a final pH of between 6.2 to 7.0.

This invention also relates to a method of administering to a mammal in need of a pharmaceutically active agent, the method comprising administering a palatable, semi-solid, spill-resistant pharmaceutical solution for oral administration comprising (a) from about 0.05 to about 5.0% of a pharmaceutically active agent; (b) from about 0.18 to about 0.35% of a carbomer; (c) up to about 50% glycerin; (d) from about 10 to about 30% polyethylene glycol; (e) up to about 2.0% sucralose liquid concentrate; and (f) water. The composition has a pH of between 6.0 to 7.2 and a viscosity of between 6,000 to 13,000 cps.

### DETAILED DESCRIPTION

In describing embodiments of the present invention, specific terminology is employed for the sake of clarity. However, the invention is not intended to be limited to the specific terminology so selected. It is to be understood that each specific element includes all technical equivalents, which operate in a similar manner to accomplish a similar purpose. The above-described embodiments of the invention may be modified or varied, and elements added or omitted, without departing from the invention, as appreciated by those skilled in the art in light of the above teachings. Each reference cited here is incorporated by reference as if each were individually incorporated by reference.

Where the term “pharmaceutical” is used herein, it should be understood to include prescription, over the counter, GRAS (generally recognized as safe), nutraceutical, and other products whether subject to approval by a drug regulatory agency or not.

Pharmaceutical formulations according to the invention comprise an agent or a pharmaceutically acceptable salt thereof as an active ingredient together with one or more pharmaceutically acceptable carriers, excipients or diluents. Any conventional technique may be used for the preparation of pharmaceutical formulations according to the invention. The active ingredient may be contained in a formulation that provides quick release, sustained release or delayed release after administration to the patient.

Useful pharmaceutical agents include analgesics (e.g. acetaminophen, codeine, aspirin and dihydrocodeinone), anti-inflammatory agents (e.g. ibuprofen, naproxen and diclofenac), anti-histamines (e.g. H₁-blockers, such as chlorpheniramine, terfenadine, loratidine, astemizole and cetirizine and H₂-blockers, such as cimetidine and ranitidine), anti-infectives (e.g. antibacterials such as sulfa drugs, i.e. sulfisoxazole, and cephalosporins, penicillins, and macrolide antibiotics; quinolones, i.e. ciprofloxacin and ofloxacin tetracyclines, i.e. tetracycline; anti-virals, i.e acyclovir and amantadine and anti-fungals, i.e. fluconozole), bronchodilators (e.g. albuterol, metaproterenol and theophylline), cough suppressants (e.g. dextromethorphan), expectorants (e.g. guaifenesin), decongestants (e.g. pseudopehedrine), CNS active agents (e.g. hypnotics, such as triazolam; sedatives, such as phenobarbital; tranquilizers, such as chlorpromazine and diazepam; antidepressants, such as fluoxetine and nortriptylline), anti-convulsants, such as carbamazepine and ethosuximide and anti-Parkinson's agents, such as L-DOPA), cardiovascular (e.g. including: diuretics, such as hydrochlorthiazide; including: beta-blockers, such as propranolol; ACE inhibitors, such as captopril and enalapril; calcium channel blockers, such as diltiazem; anti-anginals, same as anti-hypertensive agents; cardiac glycosides, such as digoxin), antineoplastics (e.g. 5-fluorouracil and cyclophosphamide), Cholesterol-lowering agents (e.g. lovastatin or simvastatin), antiemetics (e.g. metoclopramide), Vitamins (e.g. B vitamins; folic acid, vitamin A), minerals (e.g. iron, calcium and zinc salts and fecal softeners, such as docusate), plant extracts (e.g. echinacea, gingko biloba, St. John's wort, etc), and pharmaceutically acceptable salts and esters of the named compositions.

The inventive spill-resistant formulation does not contain a seaweed polysaccharide such as agar, algin, carrageenan, furcelleran or a mixture thereof.

Depending on the specific combination of components, various ranges may be used for each of the components. Exemplary amounts (w/w) of active ingredient or acetaminophen are from about 0.5 to 5.0%; about 1.0 to 3.5%; about 2.5 to 3.0%; or about 2.75%. Exemplary amounts (w/w) of neutralized carbomer are up to about 1.0%; about 0.18 to about 1.0%; 0.2 to about 1.0%; about 0.18 to 0.6%; about 0.25 to about 0.6; about 0.25 to 0.5; about 0.18 to 0.35%; about 0.25 to about 0.35%; about 0.25 to 0.29%; or about 0.27%. Exemplary w/w amounts of polyethylene glycol (PEG) are from at least about 5 to about 30%; about 5 to 20%; about 5 to 10%; or about 5, 10, 15, 20 or 25%. Exemplary amounts (w/w) for sucralose are up to about 2.0%; from about 0.2 to 2.0%; about 1.0 to 2.0%; or about 0.4%. Exemplary amounts (w/w) of glycerin are up to about 50%; about 5 to 50%; or about 35 to 50%. The composition may have approximate viscosity values (cps) from about 5000 to 20000; 5000 to 15000; 5000 to 10000; 6000 to 17000; 6000 to 13000; 7000 to 13000; 8000 to 11000; or about 7000, 8000, 9000, or 10,000. Moreover, the pH of the composition may be over about 5.5; over about 6.0; from about 6.2 to about 7.0; or up to about 7.5. Thus, compositions within the scope of the invention have at least the lower limit of the various ranges listed above, and do not exceed the various upper limits. Beyond the stated lower and higher limits for each component, formulation characteristics fall outside the desired properties of the inventive composition.

Where the term spill-resistant is used herein, it should mean a composition having the following properties. As described in Mehta et al., U.S. Pat. No. 6,071,523, incorporated herein by reference, the term “spill-resistant formulation” refers to a product which, as sold, has viscosity in a certain range (e.g. 5,000 to 20,000 cps), is a semi-solid, is easy to administer accurately, has spill-resistant consistency, is storage stable, and has mutually compatible ingredients. Viscosity can be measured using a Brookfield Viscometer with a ‘C’ spindle with Helipath movement at 20 RPM and 20-25 degrees C., or equivalent. Viscosity decreases slightly with increasing temperature.

These spill-resistant pharmaceutical formulations have a homogeneity wherein the active ingredient is uniformly dissolved in the vehicle. It may have a crystalline stability such that the active ingredient does not exhibit excessive crystalline growth or dissolution, so that the particles stay within a target particle size range. Heat-cool studies can be conducted to check for crystal growth and active dissolution.

The spill-resistant pharmaceutical formulations also may have solution stability such that the active ingredient remains dissolved indefinitely without agitation, eliminating the need to shake before administering. A semi-solid formulation of the invention can not be shaken easily, so the particles must remain dissolved or dispersed without shaking. Advantageously, there is no need to shake the inventive compositions. Solution stability results from a reduced sedimentation rate.

The spill-resistant pharmaceutical formulations may also have a Brookfield viscosity within the range of about 6000 cps to about 13000 cps at room temperature. Below about 6000 cps, formulations tend to spill too easily. Formulations exhibit desirable spill-resistant properties at a viscosity greater than about 6000 cps. The product spreads quickly at viscosity less than about 13000 cps. Thus spill resistance and spreading characteristics are desirable in this viscosity range. The viscosity of the spill-resistant solution is temperature sensitive between 15° C. and 45° C. The viscosity of the formulation increases with decrease in temperature and decreases with increase in temperatures. However, these changes in the viscosity and correlated spill-resistant characteristics are reversible, so that the original formula viscosity is obtained when temperature returns to room temperature (˜23° C.; broadly 19° C. to about 29° C.).

The inventive formulations have a spill-resistant consistency permitting the composition to be squeezed into a spoon from a container with light manual pressure, to spread and level in a spoon bowl quickly enough for accurate measurement (typically in about 1-5 seconds at room temperature), and to remain in the spoon bowl long enough to permit administration without spilling particularly under difficult circumstances such as encountered with dispensing to children, or by the elderly. Spill-resistance refers to the product's ability to withstand a series of tests that were developed to evaluate the product's spill resistance. For most formulations, spill resistance means the formulation does not spill from a teaspoon for a definite period, e.g. at least about 30 or 60 seconds on spoon inversion, about 30 or 60 seconds on spoon vibration, and about 10, 20, or 30 or from about 5 seconds to about 30 seconds on spoon tilting. Spill-resistant properties correlate with viscosity but are not directly linked, so that a composition within the target viscosity range may lack spill resistance. Spill resistance and a shaking test, tilting test and inversion test are described in U.S. Pat. No. 6,071,523. Spill resistance is related to whether the formulation passes a flow test, ensuring that dispensing and dosing to a 5.0 mL teaspoon is easy and satisfactorily accurate.

The spill-resistant formulations may have a flow quality having a non-Newtonian, pseudoplastic and time independent fluidity wherein the viscosity of the non-solid gel decreases with increasing shear rate, in which the behavior is fully reversible, and is indicative of Bingham behavior. There is a relationship between flow and viscosity.

Spill-resistant solutions are non-Newtonian and time independent fluids. Non-Newtonian refers to a fluid whose behavior departs from that of an ideal Newtonian fluid. These fluids have different viscosities at different shear rates and fall under two groups: time independent and time dependent. In contrast, for a Newtonian fluid the rate of shear in the fluid under isothermal conditions is proportional to the corresponding stress at the point under consideration. (McGraw-Hill Encyclopedia of Science & Technology, 6^{th} edition, 1987, Volume 12, pages 57-60). Time independent fluids are those for which the rate of shear at any point in the fluid is some function of the shear stress at that point and depends on nothing else. These fluids have a constant viscosity value at a given shear rate. The viscosities do not change with time. These solutions may be pseudoplastic according to a rheogram. The viscosity of the gel decreases with increasing shear rate, and the behavior is fully reversible. Pseudoplastic fluids are those that show no yield value, but the ratio of shear stress to the rate of shear, which may be termed the apparent viscosity, falls progressively with shear rate. The decrease in viscosity with an increase in shear rate is also known as shear thinning. This phenomenon of shear thinning is characteristic of solutions of asymmetric particles or solution of polymers such as cellulose derivatives. The viscosity of spill-resistant gel decreases with increasing the shear rate, e.g., increasing the spindle speed.

The spill-resistant solutions may exhibit Bingham behavior with a yield value about 156.0 D/cm². Bingham plastics exhibit a yield stress, which is the stress that must be exceeded before flow starts. Thereafter the rate-of-shear curve is linear. There are other materials that also exhibit a yield stress, but the flow curve is thereafter not linear. These are usually called generalized Bingham plastics. A Bingham flow requires an initial stress, the yield value, before it starts to flow. Once the yield value is exceeded and flow begins a Bingham fluid may display Newtonian, pseudoplastic or dilatant flow characteristics. These fluids exhibit different behavior than thixotropic fluid which are time-dependent.

The spill-resistant formulation can be understood using a general equation for Stokes' law, as follows (Pharmaceutical Dosage Forms: Disperse System, Volume 2, Marcel Dekker, Inc., New York and Basel., 1996, Pg. 152 (“Pharm. Dosage Forms Vol. 2”):

V=d²×(ρ_{S}−ρ_{L})g/18η

Wherein

V represents settling velocity,

d represents Stokes' diameter,

ρ_{S} represents density of solid,

ρ_{L} represents density of liquid,

g represents acceleration due to gravity, and

represents viscosity of liquid.

According to Stokes' law, reducing the sedimentation rate can be achieved by the following methods: (1) decreasing the particle size of the solvent, (2) minimizing the difference of the density between the solutes and the solvent (liquid phase), and (3) increasing the viscosity of the external phase. Most solution development focuses on the particle size rather than equalizing the density between the solute and the external phase. Solutions of the present invention have a unique combination of ingredients that provide an external phase with a density about equal to the pharmaceutically active agent.

The formulations of the pharmaceutical compositions described herein may be prepared by any method known or hereafter developed. In general, preparation includes bringing the active ingredient into association with a carrier or one or more other additional components, and then, if necessary or desirable, shaping or packaging the product into a desired single- or multi-dose unit.

The composition may contain additional components including, but not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; pharmaceutically acceptable polymeric or hydrophobic materials as well as other components.

Although the descriptions of pharmaceutical compositions provided herein are principally directed to pharmaceutical compositions which are suitable for administration to humans, it will be understood by the skilled artisan, based on this disclosure, that such compositions are generally suitable for administration to any mammal. Preparation of compositions suitable for administration to various animals is well understood, and the ordinarily skilled veterinary pharmacologist can design and perform such modifications with routine experimentation based on pharmaceutical compositions for administration to humans.

Suspensions, in which the active ingredient is dispersed in an aqueous or oily vehicle, and liquid solutions, in which the active ingredient is dissolved in an aqueous or oily vehicle, may be prepared using conventional methods or methods to be developed. Liquid solution of the active ingredient may be in an aqueous or oily vehicle and may further include one or more additional components such as, for example, suspending agents, dispersing or wetting agents, emulsifying agents, demulcents, preservatives, buffers, salts, flavorings, coloring agents, masking agent and sweetening agents. Oily solutions may further comprise a thickening agent. Liquid solutions of the active ingredient may be in an aqueous or oily vehicle and may further include one or more additional components such as, for example, preservatives, buffers, salts, flavorings, coloring agents, and sweetening agents.

The inventive formulations have attractive appearance, suitable texture and organoleptic (taste and mouth-feel) properties. The components are mutually compatible in that they do not interfere with the bioactivity of the pharmaceutical agent or physical properties of the vehicle, and the components do not separate and retain their properties. The pharmaceutically acceptable taste masking liquid comprises an active ingredient and a vehicle. The active ingredient is pharmaceutically active, and may be dissolved in the spill-resistant gel base. Solutions are defined as a class of materials in which one phase, a solid, is dispersed in a second phase, generally a liquid, in a mixture of two or more components that form a homogenous molecular dispersion, the composition of which can vary over a wide range (Physical Pharmacy: Pysical Chemical Principles in the Pharmaceutical Sciences, 4^{th} edition, 1993).

The bases useful in this invention are those having incorporated therein per 100 milliliters of liquid base about 5 to about 20 milligrams, preferably about 15 milligrams of polyethylene glycol having an average molecular weight of about 600 to about 1000, preferably about 1000; up to about 50 milligram of a carrier component; and about 0.25 to about 0.50 milligram of thickener. The viscosity of the solution may be between 6,000 to 10,000 cps measured using a Brookfield Viscometer with a ‘C’ spindle with Helipath movement at 20 RPM and 20-25 degrees C., or equivalent.

Polyethylene glycols (PEG) are stable, hydrophilic substances that can be used to enhance the aqueous solubility or dissolution characteristics of poorly soluble compounds. Surprisingly, when concentrations of low-molecular weight PEG from at least about 5% to about 20%, preferably less than about 15%, were added to the spill-resistant solutions, the solutions were perceived to be less bitter and sweeter than the solutions that did not include PEG. Useful low molecular weight PEG include PEG 600, 800, 900 and 1000.

To evaluate palatability, a sensory evaluation test was devised for these studies. Subjects were trained by tasting known compounds, and taught to evaluate sweetness, bitterness and flavor intensity on a 7-point intensity scale. The subjects were also taught to describe the texture of the two samples in their own words. Samples were evaluated in alternating sequence. The scale used is shown in Table 1.

TABLE 1
Intensity Scale

| | | Thresh- | | Slight | | Mod- | |
|---|---|---|---|---|---|---|---|
| | Thresh- | old | | to mod- | Mod- | erate | |
| | old | to slight | Slight | erate | erate | to high | High |
| Sweetness | 1 | 2 | 3 | 4 | 5 | 6 | 7 |
| Bitterness | 1 | 2 | 3 | 4 | 5 | 6 | 7 |

The thickener provides the necessary viscosity, spill-resistant properties such as pseudoplasticity, and to suspend the active agent. Carbomers are water soluble carboxyvinyl polymers (Merck Index 12^{th} ed., no. 1878) that can be used as thickeners in semisolid pharmaceutical formulations (see Mehta et al., U.S. Pat. No. 6,071,523). Carbomer 934P (Carbopol® 974P; Noveon, Inc., Cleveland, Ohio) is a suitable thickener or gelling agent. Suitable concentrations range up to about 1.0% or from about 0.2 to about 1.0%, and more specifically from about 0.25 to about 0.50%, w/w. Carbomer rheology supports a high yield value (Handbook of Pharmaceutical Excipients Third Ed., A. H. Kibbe (Ed.), Pharmaceutical Press, London, UK., 2000, Pg. 442, 79, 53 (“Handbook of Pharm. Excipients”)). Carbomers are slightly acidic and are neutralized in the formulation to a non-acidic pH, e.g., with sodium hydroxide. The non-acidic pH is over 5.5, or over about 6.0 and preferably in a range to about 7.2, to provide a formulation having maximal viscosity. In pH titration studies of a carbomer formulation, viscosity was highest between about pH 6.0 and 6.5. At pH 5.5 the viscosity was about 80% of peak and at pH 5.0 viscosity was only 50% of the peak. At pH 7.2 viscosity fell to about 70% of the peak. Thus, a neutralized carbomer is one which is in a formulation having at least 70% of the highest viscosity achievable for the formulation, or above about 80%, or above about 90% of the peak viscosity. It is unexpectedly advantageous to have a neutralized carbomer, which translates to a pH of about 5.5 or higher.

The carrier component primarily serves as the external phase of the suspension matching the density of the active agent, and as the liquid providing necessary flow characteristics, and also contributes other properties to the suspension. The carrier component may comprise glycerin up to about 55% or from about 35% to about 50%. Glycerin is widely used as a solvent, extractant, and preservative in a variety of pharmaceutical formulations.

Purified water makes up the bulk of the carrier component comprising from about 29 to 64% of the formulation. Water concentration can be less than about 50% w/w or even less than about 31% in certain acetaminophen formulations.

The solution may also comprise organoleptic components which impart desirable sensory characteristics to the solution, including taste, color and smell. The organoleptic component may comprise a high intensity sweetener that improves sensory appeal. These components may also include coloring agents that provide desired shades., products such as FD&C Blue #1, FD&C Red #40, or D&C Red #33. Flavoring agents such as cherry, grape, or bubble gum, and masking agents may be desirable.

The inventive pharmaceutical solution for oral administration is adapted to be used in conjunction with a device or package that makes it particularly easy to measure single dosage units of a pharmaceutical agent useful for systemic treatment and convenient to administer them orally in a semi-solid composition. These devices would particularly be suitable for administration to children and for self-administration by aging adults, and adults with motor problems. They are resistant to tampering by young children or individuals with limited mental capacity due to a childproof closure.

For example, bottles of different resin types, such as polyethylene and low-density polyethylene and different shapes can be used to deliver various spill-resistant pharmaceutical compositions. The squeezability of a 4-oz custom-made bottle made using polyethylene terephthalate material is satisfactory and permits controlled delivery of the spill-resistant pharmaceutical compositions. These vessels are squeezed perpendicular to the flow of the material. Various plugs of different architecture can also be used. The inventive formulation can be used with a variety of other packaging components.

The following examples further illustrate the invention, but must not be construed as limiting the invention in any manner.

### EXAMPLE 1

### Pseudoephedrine Formulations

Laboratory scale (1 kg to 3 kg) batches of pharmaceutical formulations were prepared by mixing glycerin, optionally propylene glycol, and polyethylene glycol in water, and if necessary, heated, to form a solution. The carbomer was dispersed in water and added to the polyol phase. Sucralose, color, flavors, and masking agents are optionally added and mixed. Pseudoephedrine HCL (Malladi Drugs and Pharmaceuticals Ltd.) was dissolved in water and added to the solution to give a final concentration of 15 mg/ml. If needed the pH was adjusted to within the 6.0 to 7.0 range with NaOH (10% w/w).

Different concentrations of PEG 1000 (5 to 15% w/w)) were added to and tested for taste masking properties of the PEG. The final formulations of the pseudoephedrine, Batches A to D, are given below in Table 2.

TABLE 2
GEL PSEUDOEPHEDRINE FORMULATIONS

| | Batch A | Batch B | Batch C | Batch D |
|---|---|---|---|---|
| Ingredients | % (w/w) | % (w/w) | % (w/w) | % (w/w) |
| Water | 38.5 | 38.5 | 43.3 | 33.3 |
| Glycerin | 50.0 | 50.0 | 50.0 | 50.0 |
| Propylene glycol | 10.0 | — | | |
| Polyethylene glycol 1000 | — | 10.0 | 5.0 | 15.0 |
| Carbomer 934P | 0.55 | 0.55 | 0.59 | 0.59 |
| Grape bubble gum flavor | 0.15 | 0.15 | 0.15 | 0.15 |
| Sucralose liquid | 0.30 | 0.30 | 0.30 0.30 | |
| concentrate | | | | |
| Initial viscosity at | 6400 | 6730 | 9490 | 10,090 |
| 23° C., cps | | | | |

### EXAMPLE 2

### Taste-Masking Effect of PEG on Pseudoephedrine Gel

A. A comparison of Pseudoephedrine Gel 10%(w/w) PEG-1000 to Pseudoephedrine Gel) 0% PEG 1000.

A taste testing study was done to compare two pseudoephedrine formulations Batch A with 0% PEG and Batch B with 10% PEG. The experiment tested whether the addition of PEG to the formulation corresponded to changes in the sensory perception of individuals. Sweetness was perceived as greater in the 10% PEG formulation by five of the six subjects. Only one panelist perceived the 0% PEG as sweeter. When the data was compiled, the average sweetness score for Batch B was 5.7 as compared to a score of 4.9 for Batch A. The bitterness of 10% PEG batch was rated markedly lower by all subjects. Indeed, two panelists found no bitterness at all in the 10% PEG batch. Average bitterness score was 1.8 for those compositions that contained 10% PEG as compared to 3.6 for those compositions with 0% PEG. 10% PEG NONSPIL™ GEL Pseudoephedrine formulation effected a sensory increase in sweetness and suppressed the perception of bitterness found in a PEG-free control.

TABLE 3
Intensity Scores for Pseudoephedrine Gel (0 and 10% PEG)

| | Batch A | Batch B | |
|---|---|---|---|
| | 0% PEG 1000 | 10% PEG 1000 | Net Difference |
| | (average sensory | (average sensory | (Batch B |
| | score n = 6) | score n = 6) | minus Batch A) |
| Sweetness | 4.9 | 5.7 | 0.8 |
| Bitterness | 3.6 | 1.8 | −1.8 |

B. A comparison of Pseudoephedrine Gel 5%(w/w) PEG-1000_ to Pseudoephedrine Gel 15%(w/w) PEG-1000.

Pseudoephedrine formulations with 5% PEG (Batch C) and 15% PEG (Batch D) were administered to six subjects for sensory appraisal.

Four out of the six subjects rated the 15% PEG batch as sweeter than the 5% PEG batch. Two of the six subjects rated Batch C as sweeter than Batch D. These variable findings are supported by the average scores (Table 4), where there is a small net difference in the sweetness scores between the two batches.

The majority of the subjects rated the 15% PEG batch lower for bitterness. One subject perceived the 5% PEG sample to be less bitter and one subject did not distinguish between the two batches. The higher PEG concentration suppressed the sense of bitterness in the formulation. There was a trend towards an increase in the perception of sweetness with an increase in concentration of PEG in pseudoephedrine formulations.

TABLE 4
Intensity Scores for Pseudoephedrine Gel (5 and 15% PEG)

| | Batch C | Batch D | |
|---|---|---|---|
| | 5% PEG 1000 | 15% PEG 1000 | Net Difference |
| | (average sensory | (average sensory | (Sample D minus |
| | score n = 6) | score n = 6) | sample C) |
| Sweetness | 5.1 | 5.3 | 0.2 |
| Bitterness | 4.4 | 2.1 | −2.3 |

### EXAMPLE 3

### Acetaminophen Formulations

Laboratory scale (1 kg to 3 kg) batches of pharmaceutical formulations were prepared. The glycerin, and/or propylene glycol, and/or polyethylene glycol were mixed, and if necessary, heated, to form a solution. The carbomer was dispersed in water and added to the polyol phase. Sucralose, color, flavors, masking agents were optionally added and mixed. Acetaminophen (Tyco Mallinckrodt Healthcare) was dissolved in water and added to give a final concentration of 160 mg/5 ml. The pH was adjusted to within the 6.0 to 7.0 range with NaOH (10% w/w).

TABLE 5
NONSPIL ™ GEL ACETAMINOPHEN 2.75% FORMULATIONS

| | Batch A | Batch B | Batch C | Batch D |
|---|---|---|---|---|
| Ingredients | % (w/w) | % (w/w) | % (w/w) | % (w/w) |
| Water | 26.12 | 30.93 | 55.73 | 30.93 |
| Glycerin | 40 | 50 | 35 | 50 |
| Propylene glycol | 25 | — | — | — |
| Polyethylene glycol 1000 | — | 15 | — | 15 |
| Sorbitol crystalline | 5 | — | 5 | — |
| Sucralose liquid | 0.4 | 0.4 | 0.4 | 0.4 |
| concentrate | | | | |
| Carbomer 934P | 0.265 | 0.29 | 0.28 | 0.29 |
| Grape flavor | 0.15 | 0.15 | 0.15 | 0.15 |
| Masking Agent | 0.2 | 0.4 | 0.4 | 0.4 |

### EXAMPLE 4

### Taste-Masking Effect of PEG on Acetaminophen NONSPIL™ GEL

A. A comparison of Acetaminophen NONSPIL™ Gel 15%(w/w) PEG-1000₁₃/0% Propylene Glycol to Acetaminophen NONSPIL™ Gel 0%(w/w) PEG-1000/0% Propylene Glycol

A taste testing study was done as described in Example 1. An acetaminophen composition with 0% PEG; 0% propylene glycol (Batch C) and an acetaminophen composition with 15% PEG; 0% propylene glycol (Batch D) were administered to six subjects for sensory appraisal. Three out of the six subjects ranked Batch D as tasting sweeter than Batch C. Three of the subjects detected no difference in the sweetness of the two samples. Five out of the six subjects rated Batch D as less bitter than Batch C. PEG tends to increase the sweetness and mask the bitterness of the NONSPIL™ GEL acetaminophen formulation. The formulation's palatability was generally improved.

The results from were compiled and the differences between the two samples for the entire group are given in Table 6.

TABLE 6
Intensity Scores for Acetaminophen Nonspil ™
Gel (0 and 15% PEG)/0% Propylene Glycol

| | Batch C | Batch D | |
|---|---|---|---|
| | 0% PEG 1000/0% | 15% PEG 1000/0% | |
| | propylene glycol | propylene glycol | |
| | (average sensory score | (average sensory | |
| | n = 6) | score) n = 6 | Net Difference |
| Sweetness | 5.2 | 6.0 | 0.8 |
| Bitterness | 3.8 | 2.4 | −1.6 |

B. A comparison of Acetaminophen NONSPIL™ Gel 15%(w/w) PEG-1000/25% Propylene Glycol to Acetaminophen NONSPIL™ Gel 0%(w/w) PEG-1000/25% Propylene Glycol

A taste testing study was done to test the acetaminophen composition with 0% PEG/25% propylene glycol (Batch A) and 15%PEG/0% propylene glycol (Batch B). This study was done to determine if other solvents could be taste-masking in the absence of PEG. Five out of six respondents ranked the PEG sample as being at least one unit higher than the propylene glycol sample. One subject found no difference between the samples in terms of sweetness. All of the subjects found the PEG sample to be noticeably less bitter than the propylene glycol sample. 15% PEG in the acetaminophen formulation taste masks the bitterness of the formulation, makes the formulation sweeter, and causes an increase in the general palatability of the sample.

TABLE 7
Intensity Scores for Acetaminophen
Nonspil ™ Gel 0% PEG/25%
Propylene glycol/15% PEG/0%
propylene glycol

| | Batch A | | |
|---|---|---|---|
| | 25% Propylene | Batch A | |
| | Glycol | 10% PEG 1000 | Net Difference |
| Sweetness | 4.3 | 5.5 | 1.2 |
| Bitterness | 5.3 | 2.4 | −2.9 |

We claim:

1. A pharmaceutical composition comprising:
(a) from about 0.5 to about 5.0% (w/w) of acetaminophen;
(b) from about 0.18 to about 0.35% (w/w) of neutralized carbomer; and
(c) a bitterness masking amount from about 5 to about 30% (w/w) of polyethylene glycol.

2. The composition of claim 1, having a pH over about 6.0.

3. The composition of claim 1, comprising less than about 15% polyethylene glycol.

4. The composition of claim 1, having a viscosity greater than about 5,000 cps.

5. The composition of claim 1, having a viscosity from about 7,000 to about 13,000 cps.

6. The composition of claim 1, wherein the composition tastes less bitter and sweeter in a taste test than an equivalent composition having water or propylene glycol substituted for PEG.

7. The composition of claim 1, having mutually compatible components.

8. The composition of claim 1, wherein the composition is free of seaweed polysaccharides.

9. The composition of claim 1, wherein the composition comprises from about 2.5% to about 3.0% (w/w) acetaminophen.

10. The composition of claim 1, wherein the carbomer is 934 P.

11. The composition of claim 1, wherein the carbomer 934P is from about 0.25% to about 0.29% (w/w).

12. The composition of claim 1, wherein the molecular weight of polyethylene glycol is selected from the group consisting of PEG 600, PEG 800, and PEG 900.

13. The composition of claim 1, wherein the polyethylene glycol is PEG 1000.

14. The composition of claim 1, further comprising up to about 50% (w/w) glycerin.

15. The composition of claim 1, further comprising up to about 2% (w/w) sucralose liquid concentrate.

16. The composition of claim 1, further comprising at least one pharmaceutically acceptable excipient selected from the group consisting of at least one food dye, masking agent, flavoring agent and antimicrobial agent.

17. The composition of claim 1, comprising from about 1.0 to about 3.5% (w/w) of acetaminophen, from about 0.25 to about 0.35% (w/w) of a neutralized carbomer, from about 5 to about 20% (w/w) polyethylene glycol, up to about 50% (w/w) glycerin, up to about 2% (w/w) sucralose liquid concentrate and up to 93.75% (w/w) water.

18. The composition of claim 1, comprising about 2.75% (w/w) acetaminophen, about 0.27% (w/w) neutralized carbomer, about 50% (w/w) glycerin, about 15% (w/w) polyethylene glycol 1000, about 0.4% (w/w) sucralose liquid concentrate, and about 31.58% (w/w) water.

19. A method comprising administering the composition of claim 1 to a mammal in need of acetaminophen.

20. An assembly comprising the composition of claim 1 contained in a device for containing and measuring a unit dose of said composition, said device comprising a sealed squeezable container, said container having an outlet, the container comprising an outer flexible squeezable wall which can be squeezed laterally with respect to an axis of said outlet whereby a predetermined unit dose of the pharmaceutical composition can be easily squeezed from the container, measured, and administered orally.

21. A process for preparing a pharmaceutical composition comprising, without regard to order, the steps of:
dispersing carbomer in a liquid to form a first solution;
dissolving acetaminophen in water to form a second solution;
heating polyethylene glycol to liquid form;
mixing polyethylene glycol into the second solution;
mixing the solution and cooling the mixture to less than 40° C.; and
titrating the mixture with a sodium hydroxide solution to a final pH of between 6.2 to 7.0.

22. The process of claim 21, wherein the carbomer is dispersed in propylene glycol until a lump free dispersion is formed.

23. The process of claim 21, further comprising mixing butylparaben into the second solution.

24. The process of claim 21, comprising heating the second solution to about 60° C. to about 70° C.

25. The process of claim 21, wherein the pharmaceutical composition comprises about 2.75% (w/w) acetaminophen, about 0.27% (w/w) carbomer, about 50% (w/w) glycerin, about 15% (w/w) polyethylene glycol 1000, and about 0.4% (w/w) sucralose liquid concentrate.

26. A method of making a taste-masking spill-resistant pharmaceutical composition comprising from about 1.0% to about 3.5% (w/w) acetaminophen and a spill-resistant base comprising from about 0.18% to about 0.35% (w/w) carbomer and from about 5% to about 30.0% (w/w) polyethylene glycol (PEG), comprising
(a) determining a bitterness masking amount of polyethylene glycol (PEG), and
(b) adding said bitterness masking amount of PEG to the spill-resistant base to form said composition for oral administration.

27. A taste-masking spill-resistant pharmaceutical composition for oral administration, comprising a pharmaceutical agent and a spill resistant base, the pharmaceutical agent or base being bitter in the absence of taste masking, the base comprising a bitterness masking component consisting essentially of polyethylene glycol (PEG) in a concentration from about 5% to about 30% w/w, and a thickener consisting essentially of neutralized carbomer in a concentration from about 0.18% to about 0.35% (w/w), wherein the pharmaceutical agent is not acetaminophen.

28. The composition of claim 27, wherein the pharmaceutically active agent is selected from the group consisting of analgesics, anti-inflammatory agents, anti-histamines, anti-infectives, bronchodilators, cough suppressants, expectorants, decongestants, CNS active agents, anti-convulsants, cardiovascular agents, antineoplastics, cholesterol-lowering agents, anti-emetics, vitamins, minerals, plant extracts and pharmaceutically acceptable salts and esters thereof.
